(19) United States
(12) Patent Application Publication
(10) Pub. No.: US 2006/0107301 A1
(43) Pub. Date: May 18, 2006

(54) Video recorder unit and method of operation therefor

(75) Inventors: Godert Willem Renswoud Leibbrandt, Eindhoven (NL); Freddy Snijder, Eindhoven (NL); Gerhardus Engbertus Mekenkamp, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics, N.V., Eindhoven (NL)

(21) Appl. No.: 10/528,490

(22) PCT Filed: Aug. 8, 2003
(86) PCT No.: PCT/IB03/03560

§ 371 Date: Mar. 18, 2005

(30) Foreign Application Priority Data
Sep. 23, 2002 (EP) 02078942.6

(51) Int. Cl. H04N 7/16; H04N 7/10; H04N 7/173; H04N 7/025

(52) U.S. Cl. 725/134; 725/139; 725/142; 725/32

(57) ABSTRACT

The invention relates to a video recorder unit which has a receiver (211) receiving mixed content video channels (101,103,105) having a mixed content of special content clips (111,117,123,127,129,133,135,139,149) and programs (107,113,119,125,131,137,138,141,145). A detection processor (213) detects the special content clips and an extraction controller (215) extracts the clips and stores them in the memory (217). A group controller (219) groups the special content clips and a play back video generator (225) plays back the group of the special content clips. In this way, the user is presented with a continuous sequence of special content clips extracted from the plurality of mixed content channels whereby a virtual channel of special content clips, such as previews or trailers, is generated.

The invention relates to a video recording unit and a method of operation therefor and in particular to a digital personal video recording unit.

In most TV broadcasts of today the TV programs are separated not only by advertising but also by station signature clips, public information broadcasts and previews for future programs.

In recent years the use of previews or trailers have increased significantly and now form a significant part of broadcasts from not only private commercial TV stations or cable networks but also for public funded channels.

Announcement by previews or trailers is now one of the primary means of announcing future programs or new series of TV programs. Other means including the traditional printed TV listing, electronic program guides and billboard announcements. However, previews have specific benefits for the broadcaster as well as the viewer. For the broadcaster, previews provide the benefits of being completely under their control, being cheap, being communicated by the same medium as the programs and thus being inherently aimed at the desired target group. Further previews have a promoting effect which can significantly increase the number of viewers for a specific program. For the viewer, previews provide the benefit of being easy to access and being highly representative of the actual program allowing for significantly more information than for example a printed program listing.

An example of a broadcast cable TV system using previews is given in U.S. Pat. No. 5,945,987. U.S. Pat. No. 5,945,987 describes an interactive network system with a video on demand application that enables viewers to select criteria for grouping various video content programs (e.g. movies, games, TV shows etc) into manageable sets for easy review. Once the programs have been grouped trailers for the programs are displayed. The trailers and programs are centrally stored on a media server database at a central headend. The headend transmits the trailers to many users If one of the many users requests a program during viewing of a trailer, the video on demand application allows the user to rent the program immediately from the trailer being shown without returning to a menu or other order screen.

However, previews are typically embedded in broadcasts without the viewer having any control. The user may therefore easily miss a preview or may have to watch previews when he is not interested in them. Simply deleting previews or omitting them from the broadcast will further result in the user missing the information on future programs contained in the previews.

Accordingly, the Invention seeks to provide an improved system for handling special content clips and preferably previews from mixed content video channels.

Accordingly, there is provided a video recorder unit comprising:

a receiver for receiving at least one of a plurality of mixed content video channels having a mixed content of special content clips and programs; memory for storing selected video signals from the received channels, a detection processor for detecting the presence of a special content clip in the at least one of the plurality of channels; an extraction controller for extracting special content clips from the at least one of the plurality of channels in response to the detection by the detection processor and storing the special content clip in the memory;

a group controller for creating a group of special content clips by grouping a plurality of special content clips extracted and stored by the extraction controller; and a play back video generator for playing back the group of the special content clips whereby the user is presented with a continuous sequence of special content clips extracted from the plurality of mixed content channels.

Hence, a user of a video recorder unit is provided with increased flexibility and control of the viewing of special content clips embedded in mixed content video channels. Not only can the special content clips thus be filtered from the content of recorded programs, but they can further be recorded and viewed at any given time, without a user specifically having to activate the recording. Further, as special content clips often are not scheduled, an automatic scanning, selection and recording of special content clips provides the user to view the special content clips without having to view the video channels for extended periods of time. In addition, a new virtual channel can be created from the special content clips which may provide additional entertainment for the viewer.

In accordance with an embodiment of the invention, the video recorder unit is a personal video recorder. Hence, a personal video recorder (PVR) is provided which provides additional flexibility and control for the end user.

In accordance with another embodiment of the invention, the play back video generator is operable to generate only one play back video signal, and the video recorder unit comprises a user input interface operable to receive a user input and the video recorder being operable to either play back the group of special content clips or programs in response to this user input. Hence, especially for e.g. a personal video recorder, the complexity and cost of the video recorder unit is maintained low while still being able to provide the control and flexibility of the viewing of special content clips.

In accordance with a further embodiment of the invention, the video recorder is operable to change the play back from a first special content clip to a corresponding program in response to the user input detecting an activation during the play back of the first special contents clip. Hence, the user is provided with the ease of operation and/or flexibility in viewing and selection of a desired program, as a program can be selected simply by an activation, e.g. a button press, during the showing of a corresponding special content clip.

In accordance with a different embodiment of the invention, the video recorder unit further comprises a controller operable to arrange at least one video recording in response to a user input selection of a special content clip and an association between the special content clip and at least a related program. Hence, the user is provided with the ease of operation and/or flexibility in recording and selection of a desired program, as a program can be selected simply by an activation, e.g. a button press, during the showing of a corresponding special content clip. This allows for a very simple setting of a timer of a video recorder, which is a task traditionally found to be very difficult by the majority of users.

In accordance with another embodiment of the invention, the special content clip is a preview of the related program and the at least one video recording is at least one video recording of the related program. Hence, the user is provided with an extremely simple method for setting the video recorder to record a program.

In accordance with a further embodiment of the invention, the related program comprises a plurality of sub programs individually transmitted on the mixed content video channels, and the at least one video recording is a recording of each of the sub programs of the related program. Hence, a very simple method of setting the recording of a plurality of programs is provided without requiring individual setting for the individual programs. As a specific example, the user can set the recorder to record all programs of a TV series simply by performing an activation during a preview of the TV series.

According to one embodiment of the invention, the special content clip is a preview clip. Hence, a close association between the special content clips and specific programs is provided thereby allowing a user with a video recorder unit wherein all previews embedded in the video channels and transmitted over a duration of time may be viewed as a virtual channel. Not only will many users find such a viewing entertaining in itself but additionally it provides a very attractive method of selecting recordings of future programs.

According to another embodiment of the invention, the extraction controller is further operable to determine if a special content clip is already stored in the memory and not to store the special content clip if so. Hence, repeated broadcasts of the same special content clips need not to use additional memory. In addition, the user is not forced to repeatedly watch identical special content clips despite these being repeatedly broadcast.

According to one embodiment of the invention, the extraction controller is operable to store an identification of each stored special content clip, the identification being derived from the video content of each stored video content clip, and detection if the special content clip is already stored is by comparison of an identification of the special content clip with the identifications stored. This provides for a simple, fast and storage efficient method of determining if the special content clip has been stored previously.

According to another embodiment of the invention, the detection processor is operable to detect that a special content clip is being received from information data comprised in the video transmission. This provides a low complexity, yet reliable and fast method of determining if a special content clip is being transmitted.

According to a further embodiment of the invention, the detection processor is operable to detect that a special content clip is being received from content analysis of the received video signal. Hence, this allows for detection of special content clip without requiring the presence of dedicated information indicating that a special content clip is being transmitted.

According to a different embodiment of the invention, the play back video generator is operable to play back a selected sub group of the group of special content clips in response to a user profile. Thus, the user can be presented with special content clip specifically suited for his personal taste or viewing habit.

According to a further embodiment of the invention, the play back video generator is operable to play back the group of special content clips in an order dependent on previous viewing of special content clips of the group of special content clips. Hence, the ordering can be specifically ordered to suit a user rather than simply being in chronological order. For example, the play back may be in the order of the most recently received previously unviewed special content clips followed by older previously viewed content clips.

According to another embodiment of the invention, the play back video generator is operable to play back the group of special content clips in an order dependent on a user preference profile. Hence, the order of the play back may specifically be suited for the user.

According to a different embodiment of the invention, the video recorder unit further comprises a remote control having a special content clip activation user input, and wherein the video recorder unit is operable to begin play back of the special content clips upon activation of the special content clip activation user input. Hence, a very user-friendly method of controlling play back of the special content clips is provided.

According to a another embodiment of the invention, the video recorder unit is operable upon activation of the special content clip activation user input to: if the group of special content clips is being played back, set a recording arrangement for a program corresponding to a special content clip currently being played, and if the group of special content clips is not being played back, begin the play back of the group of special content clips. Hence, a user is preferably provided with a very simple mechanism for selecting programs for recording or initiating the play back of special content clips.

According to another embodiment of the invention, the video recorder unit is operable upon activation of the special content clip activation user input during play back of the group of special content clips to: start play back of the next special content clip of the group of content clip for a shorter activation of the special content clip activation user input; and set a recording arrangement for a program corresponding to a special content clip currently being played for a longer activation of the special content clip activation user input. Hence, a very user-friendly method for navigating through the play back of special content clips and selecting programs for recording is provided.

According to a second aspect of the invention, there is provided a method of operation for a video recorder comprising the steps of:

receiving a plurality of mixed content video channels having a mixed content of special content clips and programs; storing selected video signals from the received channels in a memory, detecting the presence of a special content clip in at least one of the plurality of channels; extracting special content clips from the mixed content channels in response to the detection of the presence of a special content clip and storing the special content clip in the memory; creating a group of special content clips by grouping a plurality of extracted and stored special content clips; and

playing back the group of the special content clips whereby the user is presented with a continuous sequence of special content clips extracted from the plurality of mixed content channels.

These and other aspects of the invention will be apparent from and elucidated with reference to the embodiments described hereinafter.

An embodiment of the invention will be described, by way of example only, with reference to the drawings, in which

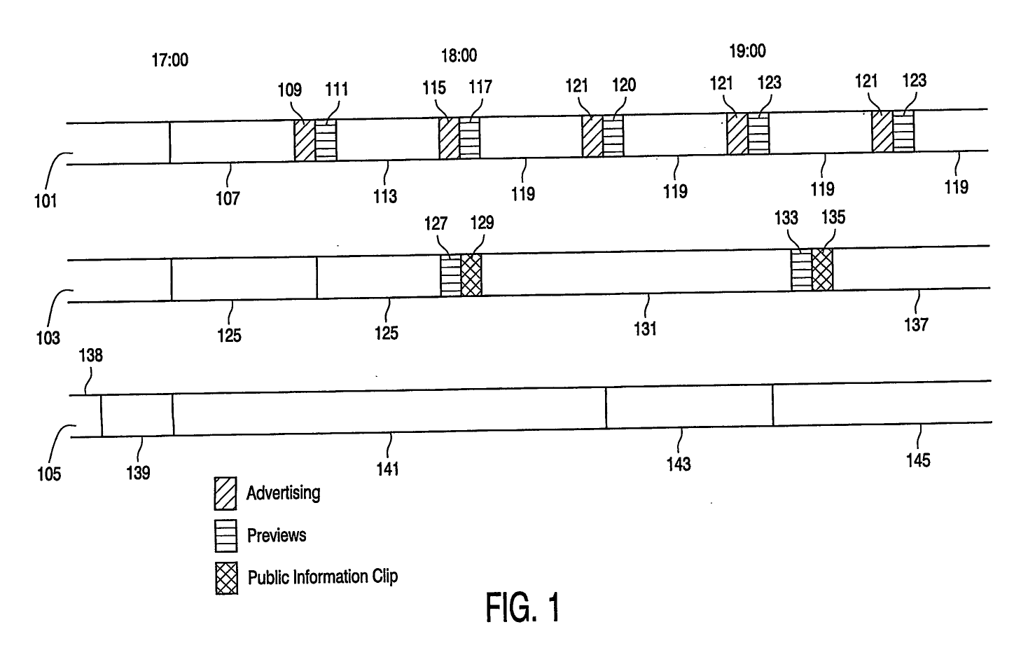

FIG. 1 is an illustration of an example of a timing diagram for video signals from a broadcaster;

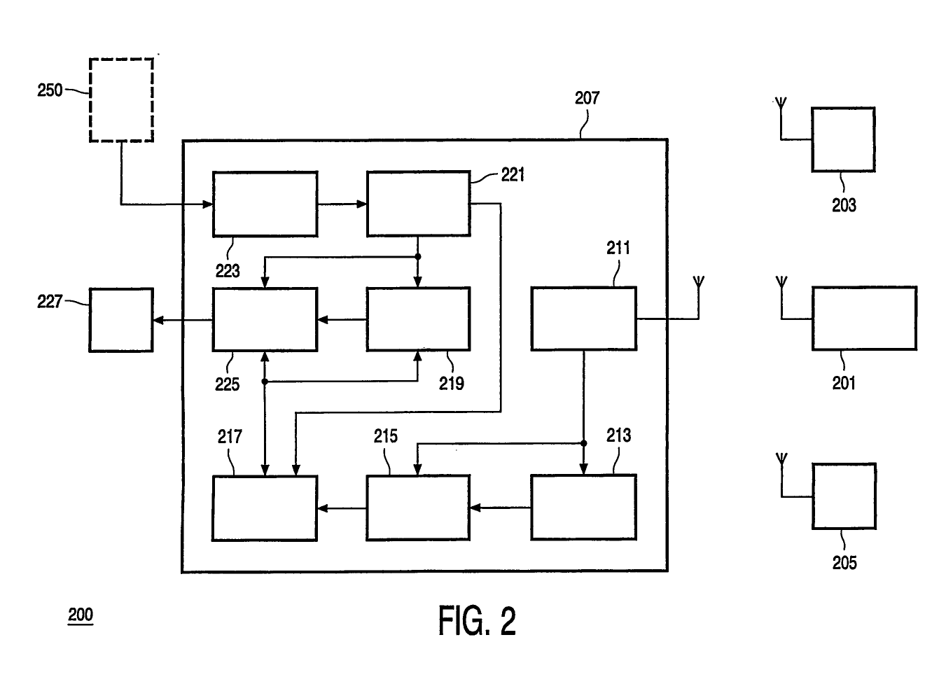

FIG. 2 is an illustration of a video broadcast and reception system comprising a video recorder unit in accordance with a preferred embodiment of the invention;

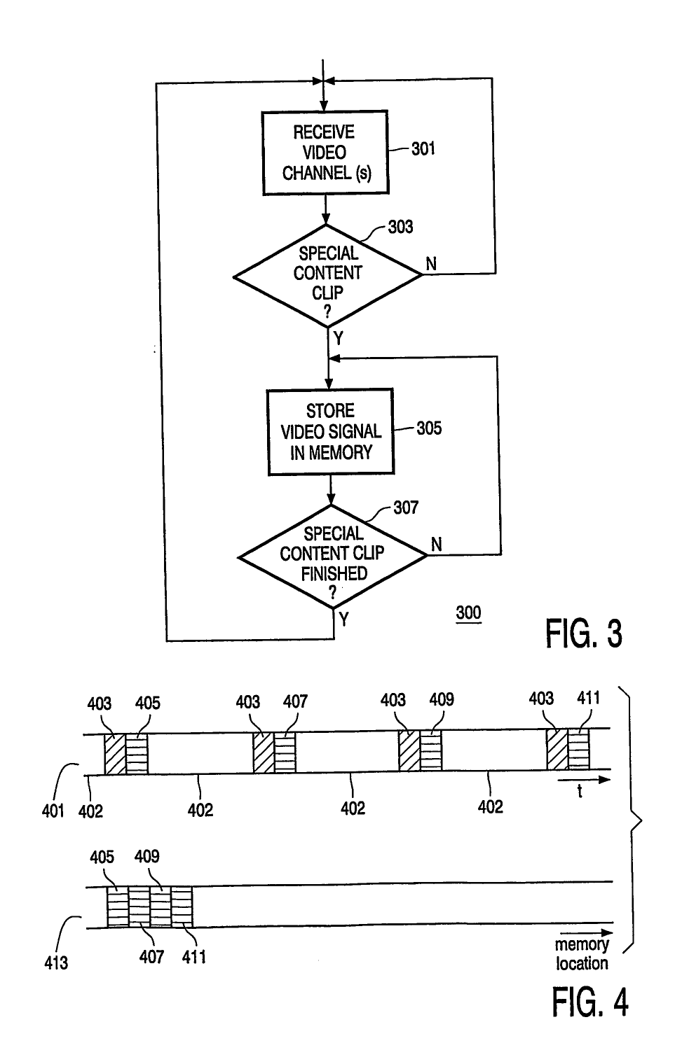

FIG. 3 illustrates a method of operation 300 for the video recorder unit 207 in accordance with an embodiment of the invention;

FIG. 4 is an illustration of an example of grouping of special content clips in accordance with an embodiment of the invention; and

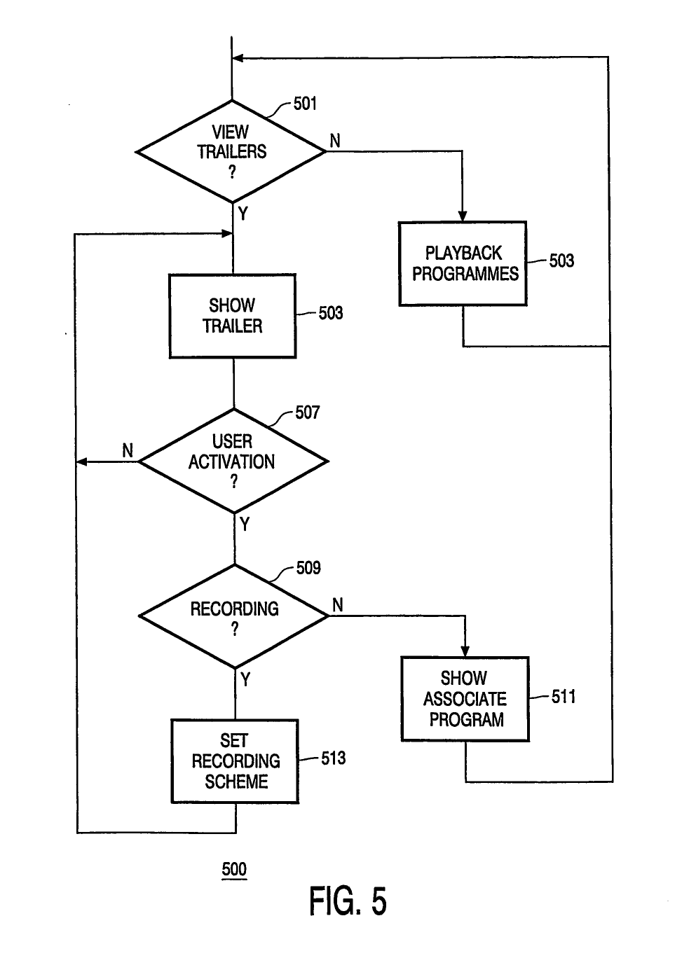

FIG. 5 shows a method of playing back special content clips in accordance with an embodiment of the invention.

FIG. 1 is an illustration of an example of a timing diagram for video signals from a broadcaster. The broadcaster in the present case transmits three video channels 101, 103 and 105. All three video channels have mixed content and transmit a combination of programs and special content clips. Programs include films, documentaries, news programs, game shows, dramas, comedy shows etc and tend to be of longer duration, typically from 10 minutes and upwards. The special content clips tend to be of short duration, typically less than around 5 minutes. The special content clips tend to not be advertised in themselves but are aimed at providing specific information, previews or at promoting for example programs, films, views and/or products. As such, special content clips may be public information broadcasts, advertising news flashes etc. Especially, the special content clips may be previews or trailers related to programs shown by the broadcaster, including previews of future programs, films or series. The preview may be of programs on other channels or even of programs of other broadcasters.

As seen in the example of FIG. 1, a first video channel, such as a TV channel, 101 (channel 1) shows a news program 107 on the day shown. Following the news program 107, a short special content clip in the form of a short advertisement block 109 is shown followed by another special content clip in the form of a preview 111 of a future program is shown. This is followed by an episode of the TV comedy “Friends” 113 after which another short advertisement block 115 and preview block 117 follows. Further programmes 119 follow each separated by short special content clips in the form of advertisement blocks 121 and preview blocks 123.

A second video channel, channel 2, 103 is a subscriber finded pay channels which does not comprise any advertising. At 17.00 channel 2 103 shows an program in the form of an episode of the TV comedy “Frasier” 125 followed immediately by another program in the form of an episode of the cartoon “The Simpsons” 126. This program is followed by a special content clip in the form of a preview 127 of for example a future program or series of programs to be shown on Channel 2 103. The preview 127 is followed by a different form of special content clip in the form of an information clip 129 which provides suitable information to viewers. The information can be any suitable information and is preferably aimed specifically at the target group for the current channel. Thus, the information clip may comprise a short news flash or may be more specifically aimed information such as for example the current share prices and index, transport information, sports result update (e.g. the latest cricket or football score including clips of the highlights which have occurred during the preceding program). The information clip 129 is followed by another program in the form of the film “Casablanca” 131. Further previews 133, information clips 135 and programs 137 follow. Channel 2 is an example of a subscription channel wherein the content may be specifically aimed at a target group, such as sports enthusiast, wherein the special content clips can be specifically aimed at the interests of that group.

A third video channel, channel 3, 105 is a subscription film channel wherein films are shown in their entirety without interruption. In this example, films start only on the hour and the time between the ending of a film and the beginning of a new filn is filled up with previews of future presentations. Thus a film “Saving Private Ryan” 138 finishes some time before 17.00 and the interval 139 between the ending of the film 138 and 17.00 is filled up with a plurality of special content clips in the form of previews. The number of previews depends on the length of the interval 139. At 17.00 the next program in the form of the film “Star Wars” 141 begins followed by another interval 143 filled with previews until the start of the next film “The Deer Hunter” 145.

FIG. 2 is an illustration of a video broadcast system 200 comprising a video recorder unit in accordance with a preferred embodiment of the invention.

The video broadcast system 200 comprises a broadcast transmitter 201 which broadcasts a number of video channels to a plurality of users. One example of a suitable broadcast system is a TV broadcast system. Hence, in the embodiment shown, the broadcast is by means of radio signals but it is within the contemplation of the invention that any suitable means can be used including for example cable broadcast techniques. The requirements and design of suitable broadcast systems for distributing video signals are known by the person skilled in the art and will for brevity not be discussed further here.

The broadcast transmitter thus transmits a plurality of mixed content video channels having a mixed content of special content clips and programs. The special content clips are embedded in the video signals for the video channels as described previously.

It will be understood that the video signals transmitted by the broadcast transmitter also comprise audio signals for the soundtrack accompanying the video images. Thus, video signal in the preferred embodiment denotes the combined video and audio signals.

The broadcast system comprises a number of video recorder units 203, 205, 207 which receive the video signals broadcast from the broadcast transmitter 201. The video recorder units are preferably personal video recorders (PVR). In addition, other units, such as for example TVs or video monitors, may also receive the broadcast video signals.

FIG. 2 shows one of the video recorder units in greater detail. The video recorder unit 207 comprises an antenna 209 for receiving the radio signals from the broadcast transmitter. The antenna 209 is connected to a receiver 211, which receives at least one of the plurality of broadcast video channels and generates a demodulated video signal which in the preferred embodiment is a digital video signal compressed according to the MPEG-2 compression algorithm.

The receiver 211 is connected to a detection processor 213 for detecting the presence of a special content clip in the digital video signal. In the preferred embodiment, meta data embedded in the digital video signal is used to determine that the current contents of the video signal is a special content clip.

The receiver 211 is further connected to an extraction controller 215 which is also connected to the detection processor 213. When the detection processor 213 detects the presence of a special content clip, it controls the extraction processor 215 to extract the special content clips from the digital video signal. Further, the extraction controller 215 is connected to a memory 217 for storing the special content clip. The memory 217 is preferably a hard disk and in addition to the extracted special content clips it is used to store programs and other video signals for late viewing. Hence, the same memory 217 is in the preferred embodiment used of both the storage of special content clips and the storage of normal video recordings to provide the time shifting of programs as in conventional video recording units.

The video recorder unit 207 further comprises a group controller 219 for creating a group of special content clips by grouping a plurality of special content clips extracted and stored by the extraction controller. In the preferred embodiment, the group controller 219 for example groups all the special content clips extracted together. The grouping may be a physical grouping of clips in the memory 217 or may be a logical grouping by any suitable association of a plurality of special content clips including a grouping of pointers to memory locations of the memory 217 or other links between clips.

In the preferred embodiment, the video recorder unit 207 comprises a video recorder controller 221 which is responsible for controlling all functional blocks of the video recorder unit 207. Specifically, the video recorder controller 221 is connected to a user interface 223 and is operable to control recording and play back of the video recorder in response to user inputs received through the user interface 223.

The video recorder unit 207 furthermore comprises a play back video generator 225 connected to the video recorder controller 221, the group controller 219, and the memory 217. The play back video generator 225 comprises finctionality for generating a suitable video signal from the stored video signal. The generated video signal is then fed to a display 227, which in the preferred embodiment is external to the video recorder unit 207, and which may for example be a monitor or TV. The generated video signal is in the preferred embodiment identical to a video signal being retrieved from the memory 217. However, in some embodiments, some processing may occur such as for example UHF modulation of the stored video signal to render it suitable for being fed to a TV through standard aerial connection. In other embodiments, conversion from a digital to an analogue video signal may be implemented.

In response to a user input requesting viewing of special content clips, the video recorder controller 221 controls the play back video generator 225 to play back the group of special content clips such that the user is presented with a continuous sequence of special content clips extracted from the plurality of mixed content channels. The video recorder controller 221 may directly control the operation or may for example do so through the group controller 219. Likewise, the play back video generator 225 may receive the retrieved special content clip video signal through the group controller 219 or directly access the memory 217 itself.

FIG. 3 illustrates a method of operation 300 for the video recorder unit 207 in accordance with an embodiment of the invention.

In step 301 the receiver 211 receives at least one of the channels being broadcast by the broadcast transmitter 201. In step 303, the detection processor 213 detects if the received video signal currently is a special content clip such as a preview (trailer). If so, the method 300 proceeds in step 305 and otherwise it returns to step 301.

The detection processor 213 can use different methods for detecting whether the current signal being received is a program or a special content clip. In accordance with the preferred embodiment, the video signal is a digital video signal which comprises information related to the content of the video signal. Thus in the preferred embodiment, the video signal comprises meta data which indicates for example whether the current video content is a film, a news program, a sports program, a preview (trailer), adverts etc. Hence, specifically the meta data comprises information that can directly be used to determine if the content is special content clip or a program. For example, the specification known as TV—anytime defines a format for this type of meta-data and identifies content using content reference identifiers. The detection processor 213 thus simply decodes the meta data of the video signal, and if the meta data indicates that the content is within one of the groups that the user has defined to be a special content clip, it provides this indication to the extraction controller 215.

In a more complex embodiment, the detection processor 213 is able to detect the category of the video content by a content analysis of the video signal of the video channel. In this case the detection processor 213 analyses the signal and determines if certain characteristics are indicative of the video signal being a program, a preview, advertising etc. Specifically, the analysis may include determining characteristics such as audio volume level, frequency of scene changes, etc and from a comparison between these characteristics with known typical values for the different categories, a detection of the presence of special ontent clips can be achieved.

Information obtained from this analysis highlights the differences in normal content and e.g. previews or trailers. Techniques for deciding whether a video fragment could be a preview are similar to known techniques for detecting commercials. Strong indicators for previews are a large amount of scene changes, appearance and disappearance of station logo's, and recognition of introduction frames. Clues like the encoding format or bit-rate when detected are significant for determining preview boundaries. Additionally beginings and ends of TV programs can be detected to find time slots where previews are likely to be present. An important source of information from a preview will be text and speech. Text can be obtained from closed captioning or teletext subtitling when available. Additionally overlaid text in the video can be detected, based on the amount of horizontal and vertical edges, and segmented out. Using optical character recognition techniques, these graphics-texts can be converted in to machine readable text. Additionally speech recognition or word spotting can be used to obtain machine readable text from the audio track. Text analysis is then used to look for important clues such as “Tomorrow at BBC 2”. This analysis can be based on lists of frequently occurring words and phrases. Matching names and dates mentioned to an Electronic Program Guide provides clues for which program the preview is intended.

In step 305, the method continues by storing the video signal of the special content clip in the memory 217.

In step 307, it is determined if the special content clip has finished. If not the method proceeds in step 305 and continues to store the video signal. If the special content clip has finished, the method returns to step 301 and continues to receive the video channel.

In this way, the video recorder unit continually receives and scans the received video channel for special content clip. Whenever one is detected it is stored in the memory 217. Further, the video recorder unit 207 may automatically change channels and as such may monitor a first video channel for a given duration followed by a second video channel for a given duration. Hence, embedded special content clip of one or more mixed content video channels can automatically be extracted and stored in the memory 217.

In accordance with a simple embodiment of the invention, all detected special content clips are stored in the memory. However, in the preferred embodiment, a more complex algorithm is used wherein it is determined if the detected special content clip has already been stored. If so the current special content clip overwrites the existing version thereby ensuring that the most up to date version of the special content clip is stored in memory. Alternatively, the current special content clip may be skipped and the already stored clip may be retained.

In yet another embodiment, it is detected if the current special content clip is already related to a special content clip currently stored, and if so the related special content clips are grouped together. In this way previews of the same film or news flashes relating to the same incident may be grouped together.

In the preferred embodiment, the extraction controller 215 is operable to store an identification of each stored special content clip. The identification is derived from the video content of each stored video content clip and detection if the special content clip is already stored is by comparison of an identification of the special content clip with the identifications stored.

The identification may for example be generated by well-known techniques for video fingerprinting. Such techniques typically compute a key based on the video content.

A typical method of fingerprint generation is to take a small sample of the clip (e.g. 5 seconds) and register the sequence of one or several key low-level features, like luminance mean absolute difference between subsequent frames (that is the difference between each pixel in one frame and the corresponding pixel in the next frame) etc. This sequence is the unique fingerprint. Further information can be found in for example Arun Hampapur et. al: Comparison of sequence matching techniques for video copy detection, Proceedings of SPIE Vol. 4676 (2002), pp 194-201)

The identification is preferably unique for each video content or the number of possible identifications is so high that the probability of two different clips having identical identifications is very low. As each new special content clip is stored, the identification is also stored separately in memory 217 (or possibly in a different memory) whereby a list of stored video clip identifications is built up. As a new special content clip is received, the identification is determined. The list of identifications is then searched for a match and if none is found, the special content clip is stored in the memory 217.

FIG. 4 is an illustration of an example of grouping of special content clips in accordance with an embodiment of the invention.

In the example, a first channel 401 comprises programs 402 separated by blocks of adverts 403 and previews 405, 407, 409, 411. In the example, a user input has indicated that the previews are the special content clips which should be extracted and stored. The detection processor 213 monitors the video signal as it is received, and when a preview is detected it informs the extraction controller 215 to begin to store it in the memory 217. The structure 413 of the memory is shown in FIG. 4. In the present example, the memory 217 has a block reserved for storing special content clips. As the detection processor 213 detects the beginning of a preview 405, the extraction controller begins storing the video signal in the special content clip block of the memory structure. When the detection processor detects that the preview 405 has finished, the recording is stopped. When the detection processor 213 detects the beginning of the next preview 407, the extraction controller 215 proceeds to store the clip 407 immediately after the previously stored preview 407. Similarly, when the next previews 409, 411 are received these are stored consecutively immediately following the previously stored clips 405, 407.

This mechanism provides for a very simple method of storing special content clips such that they can easily be played back in a continuos fashion. In the shown example continuous play back is simply achieved by retrieving the stored video signal beginning at the start of the preview block.

However, it is within the contemplation of the invention that any suitable method of storing the video signals in memory, grouping the signals and/or playing back a continuos sequence of special content clips. As such, the special content clips may be individually stored and grouped by logical associations of the memory locations.

FIG. 5 shows a method of playing back special content clips in accordance with an embodiment of the invention.

In step 501 a user input is checked to determine whether the user wants to view a program or to view the play back of the special content clips. If the user wants to view a recorded program, the method continues in step 503 by showing a preview which as the method initiates is the first preview. In step 507, the video recorder controller 221 determines whether there is a user input during the play back of the preview and if not the method loops back to step to step 503 and continues to play back the preview. When the current preview finishes, the play back of the following preview is started. When the last preview of the group has been played back, the play back of a preview in step 503 becomes of the first preview of the group.

If it is determined in step 507 that a user activation has occurred in step 507 the user input is evaluated. For brevity, in the shown example, the user activation can only be to instigate play back of an associated program or for setting a video recording. However, in many embodiments, the user activation may further be to finish play back of special content clips, deleting special content clips, switching off the video recorder unit etc.

In the given example, the method of play back continues in step 509 when a user activation has been determined in step 507. In step 509, it is determined if the user activation is directed to instigating a recording or a viewing of the program associated with the currently shown preview. If the user activation is for the viewing of the associated program, the method 500 continues in step 511 by identifying the program of the preview, and if the associated program has already been recorded, play back of the associated program begins. If the associated program has not been recorded earlier, a message is displayed to the user. Following step 511 the method returns to step 501.

If the user activation is directed to setting a recording scheme for the associated program of the current preview, step 513 follows step 509. In step 513, the video recorder unit is programmed to record the associated program of the preview. Hence, in the preferred embodiment, the preview comprises embedded data including details of the time and channel of the broadcast of the associated program and programs and this data is extracted and used to set the video recording. Any technique for setting the video recorder to record the associated program may be used without detracting from the invention. In some cases, the preview may be for a plurality of programs, such as a TV series or of related sport events, and the recording scheme may accordingly be set for recording of all or some of these programs.

An associated program is typically the specific program that a preview is a preview of. However, in some embodiments the association may be indirect. For example, the preview may be of a first program of a series and the associated program is the next program of that series to be broadcast or indeed the associated program may be a plurality of programs, such as all programs of the series.

In the preferred embodiment of the invention, the play back video generator is operable to generate only one play back video signal. Most current personal video recorders are designed such that only one recorded video signal can be seen at a time in order to reduce complexity and thereby cost. In this embodiment, the user input interface can receive a user input which switches between play back of the group of special content clips and play back of programs. Thus, for example, a simple user input can switch between the playing of a recorded program and the playing of the groups of special content clips.

In the preferred embodiment, the user can further select a subgroup of the group of special content clips. Preferably, this subgroup is selected depending on a user profile. The user profile is preferably determined from the users viewing habits for example by monitoring the users viewing of programs in predefined categories. In this case the storage and/or play back of special content clips is limited to special content clips belonging to categories frequently monitored. Again this analysis and selection may preferably be based on embedded data related to the content of the various special content clips and programs.

Further, in the preferred embodiment, the order of the play back of the special content clips is not necessarily in the order in which they were received or stored. Rather, the video recorder unit may be set up to play back the special content clips in an order dependent on previous viewing of special content clips of the group of special content clips. Thus, the video recorder unit monitors the user's viewing habits of the special content clips and prioritizes the play back accordingly. Thus, if the viewer frequently views special content clips related to sport events, but frequently skips previews of film and even more frequently previews of game shows, the video recorder unit will first play back all special content clips related to sport events followed by the special content clips related to films and only thereafter any special content clips related to game shows.

In the preferred embodiment, the video recorder unit 207 is operable with a remote control 250 enabling a user to control the video recorder unit at a distance. The remote control 250 has a special content clip activation user input preferably in the form of a single button denoted “special content clip” or “previews”. The playback of the stored special content clips may be started by pressing the special content clip button.

In the preferred embodiment, the function of the special content clip button depends on whether play back of the special content clips is ongoing. Hence, in one embodiment, if the group of special content clips is being played back, pressing the special content clip button will result in the video recording unit setting a recording arrangement for a program corresponding to a special content clip currently being played. For example, if a preview of a film to be broadcast later is currently being played, pressing the special content clip button results in the timer of the video recorder unit being set to record the film when it is broadcast. If the special content clip button is pressed when the video recorder unit is not playing back special content clips, the playing back of the group of special content clips will be started.

In the preferred embodiment, the action of the special content clip button during play back further has two functions. Thus, if the button is pressed shortly the play back of the next special content clip will begin and thus the remaining part of the special content clip will be skipped. If the button is pressed and held down for a longer duration the video recorder unit will instead set a recording arrangement for a program corresponding to a special content clip currently being played. For example, as described above the timer of the video recorder unit may be set to record a film the preview of which is currently being played back. In the simplest embodiment, a duration threshold is set at for example 2 seconds and if the button is pressed for less than the threshold, the play back will skip to the next special content clip and if it is longer than this threshold, the recording will be set.

The determination and/or the selection of special content clips to store/group and/or receive can be made in any suitable fashion. Thus, in some embodiments the broadcaster will define appropriate special content clips and categories of special content clips. In other embodiments, the user can select, determine and categorize programs and special content clips himself. In other embodiments the categorization and selection criteria are predefined in the video recorder unit and in yet other embodiments a combination of the previous is implemented.

In the preferred embodiment, the recording and play back of special content clips is performed independently and at different times. Thus the monitoring, detection and recording of special content clips, e.g. in accordance with the method of FIG. 3 is performed whenever the video recorder unit is idle, i.e. is not playing back or recording. However, in other embodiments the processes may be performed in parallel and /or at any suitable time. Hence, for example in multiple tuner video recorder units, one or more channels may be monitored and the special content clips recorded while a program on another video channel is recorded. In some embodiments, the play back and monitoring and recording is performed simultaneously.

In one embodiment, special content clips are scheduled similarly to the scheduling of programs, such that it is known when special content clips will be broadcast. In this embodiment, the detection processor 213 may simply detect the presence of a special content clip from the time of the broadcast. In this embodiment, the video recorder unit 207 treats the recording of the special content clips as recording of any other program except that the recording is automatic without requiring any user action or setting of the recording. The recording of the special content clips is furthermore performed at the lowest priority such that any other conflicting operation of the video recording unit 207 will take precedence and typically the recording of special content clips will only take place if no other program is being recorded.

The invention can be implemented in any suitable form including hardware, software, firmware or any combination of these. However, preferably, most finctional blocks are implemented as software program running on one or more data processors of the video recorder unit. The functionality, elements and components may be implemented in a single unit, in a plurality of units or as part of other functional units, but are preferably implemented in a single unit.

Although the present invention has been described by reference to the specific example of the preferred embodiment, it is not intended to be limited to the specific form set forth herein. Rather, the scope of the present invention is limited only by the accompanying claims.

The invention can be summarized as follows:

the invention relates to a video recorder unit which has a receiver (211) receiving mixed content video channels (101,103,105) having a mixed content of special content clips (111,117,123, 127,129,133,135,139,149) and programs (107, 113, 119,125,131,137, 138,141, 145). A detection processor (213) for detects the special content clips and an extraction controller (215) extracts the clips and store them in the memory (217). A group controller (219) groups the special content clips and a play back video generator (225) plays back the group of the special content clips. In this way, the user is presented with a continuous sequence of special content clips extracted from the plurality of mixed content channels whereby a virtual channel of special content clips, such as previews or trailers, is generated.

1. A video recorder unit comprising:
a receiver for receiving at least one of a plurality of mixed content video channels having a mixed content of special content clips and programs;
a memory for storing selected video signals from the received channels,
a detection processor for detecting the presence of a special content clip in the at least one of the plurality of channels;
an extraction controller for extracting special content clips from the at least one of the plurality of channels in response to the detection by the detection processor and storing the special content clip in the memory;
a group controller for creating a group of special content clips by grouping a plurality of special content clips extracted and stored by the extraction controller; and
a play back video generator for playing back the group of the special content clips whereby the user is presented with a continuous sequence of special content clips extracted from the plurality of mixed content channels.

2. A video recorder unit as claimed in claim 1 wherein the video recorder unit is a personal video recorder.

3. A video recorder unit as claimed in claim 1 wherein the play back video generator is operable to generate only one play back video signal, and the video recorder unit comprises a user input interface operable to receive a user input and the video recorder being operable to either play back the group of special content clips or programs in response to this user input.

4. A video recorder unit as claimed in claim 3 wherein the video recorder is operable to change the play back from a first special content clip to a corresponding program in response to the user input detecting an activation during the play back of the first special contents clip.

5. A video recorder unit as claimed in claim 1 wherein the video recorder unit further comprises a controller operable to arrange at least one video recording in response to a user input selection of a special content clip and an association between the special content clip and at least a related program.

6. A video recorder unit as claimed in claim 5 wherein the special content clip is a preview of the related program and the at least one video recording is at least one video recording of the related program.

7. A video recorder unit as claimed in claim 5 wherein the related program comprises a plurality of sub programs individually transmitted on the mixed content video channels, and the at least one video recording is a recording of each of the sub programs of the related program.

8. A video recorder unit as claimed in claim 1 wherein the special content clip is a preview clip

9. A video recorder unit as claimed in claim 1 wherein the extraction controller is further operable to determine if a special content clip is already stored in the memory and not to store the special content clip if so.

10. A video recorder unit as claimed in claim 9 wherein the extraction controller is operable to store an identification of each stored special content clip, the identification being derived from the video content of each stored video content clip, and detection if the special content clip is already stored is by comparison of an identification of the special content clip with the identifications stored.

11. A video recorder unit as claimed in claim 1 wherein the detection processor is operable to detect that a special content clip is being received from information data comprised in the video transmission.

12. A video recorder unit as claimed in claim 1 wherein the detection processor is operable to detect that a special content clip is being received from content analysis of the received video signal.

13. A video recorder unit as claimed in claim 1 wherein the play back video generator is operable to play back a selected sub group of the group of special content clips in response to a user profile.

14. A video recorder unit as claimed in claim 1 wherein the play back video generator is operable to play back the group of special content clips in an order dependent on previous viewing of special content clips of the group of special content clips.

15. A video recorder unit as claimed in claim 1 wherein the play back video generator is operable to play back the group of special content clips in an order dependent on a user preference profile.

16. A video recorder unit as claimed in claim 1 the video recorder unit further comprising a remote control having a special content clip activation user input, and wherein the video recorder unit is operable to begin play back of the special content clips upon activation of the special content clip activation user input.

17. A video recorder unit as claimed in claim 16 wherein the video recorder unit is operable upon activation of the special content clip activation user input to:
if the group of special content clips is being played back, set a recording arrangement for a program corresponding to a special content clip currently being played, and
if the group of special content clips is not being played back, begin the play back of the group of special content clips.

18. A video recorder unit as claimed in claim 16 wherein the video recorder unit is operable upon activation of the special content clip activation user input during play back of the group of special content clips to:
start play back of the next special content clip of the group of content clip for a shorter activation of the special content clip activation user input; and
set a recording arrangement for a program corresponding to a special content clip currently being played for a longer activation of the special content clip activation user input.

19. A video broadcast system comprising at least one video recorder in accordance with claim 1.

20. A method of operation for a video recorder comprising the steps of:
receiving a plurality of mixed content video channels having a mixed content of special content clips and programs;
toring selected video signals from the received channels in a memory
detecting the presence of a special content clip in at least one of the plurality of channels;
extracting special content clips from the mixed content channels in response to the detection of the presence of a special content clip and storing the special content clip in the memory;
creating a group of special content clips by grouping a plurality of extracted and stored special content clips; and
playing back the group of the special content clips whereby the user is presented with a continuous sequence of special content clips extracted from the plurality of mixed content channels.
